(19) United States
(12) Patent Application Publication
(10) Pub. No.: US 2005/0020889 A1
(43) Pub. Date: Jan. 27, 2005

(54) Medical monitoring system

(76) Inventors: Dennis P. Garboski, Bedford, NH (US); Normand N. Martel, E. Kingston, NH (US)

Correspondence Address: LAMBERT & ASSOCIATES, P.L.L.C., 92 STATE STREET, BOSTON, MA, 02109-2004, US

(21) Appl. No.: 10/899,239

(22) Filed: Jul. 26, 2004

Related U.S. Application Data
(60) Provisional application No. 60/489,771, filed on Jul. 24, 2003.

(51) Int. Cl. A61B 5/00

(52) U.S. Cl. 600/300

(57) ABSTRACT

A cost-effective, lightweight, durable, and high-quality Holter cardiac monitor with extensions enabling a single device to also provide loop recorder and event recorder functions. The system provides near real-time patient monitoring, twenty-four hours a day, seven days a week, anytime, anywhere, and is always ON. The patient worn device is one component of a complete system. The device portion of the system combines ease of use, excellent battery life, low cost, and the best features of telementry, Holter recorders, and event/loop recorders while eliminating the hassles of hookup, lost/overwritten data, and user intervention required to send in data.

### CROSS-REFERENCE TO RELATED APPLICATIONS

This application claims the benefit of U.S. Provisional Application No. 60/489,771 filed on Jul. 24, 2003.

### BACKGROUND OF THE INVENTION

1. Field of the Invention

This invention relates generally to the field of medical monitoring devices and more particularly to a patient-worn, physiological parameter monitoring, recording and transceiving device.

2. Description of the Related Art

The need for the measurement of health condition output is well established. Volumetric blood flow is clearly an important, if not the most important measurement in circulation. The need for this measurement is well documented in the medical literature by the voluminous amount of work directed at discovering a tractable method of achieving the result.

Much of the heart research conducted today is directed toward the many clinical aspects of cardiac disease. That is, such research is directed toward uncovering information that will lead to lowering the risk of heart attaches, surgical correction of heart defects and abnormalities, and restoration of the heart patient to an active lifestyle.

Cardiac monitoring is very important when a physician suspects that a patient has a cardiac problem, but can not detect any irregular cardiac symptoms in the office or hospital. There are generally two methods for monitoring cardiac outputs. The first type of cardiac monitor is known as a Holter recorder, used for the continuous recording of a patient's cardiac output. The second type of cardiac monitor is a loop, recorder. The loop recorder does not continuously store data; rather, it only stores a two minute record when a certain condition occurs; whether it be a prompt from a patient, or the occurrence of a designated threshold value. For example, when a patient senses an event or abnormal condition coming on, the patient may press an event button so that a cardiac reading can be sotred while the patient experiences this condition or event.

Patient monitors and medical monitoring systems also include monitors designed for in-patient monitoring and telemetry systems, and offer the monitoring of a variety of physiological parameters and other health information. The telemetry group of systems is aimed at short-haul and local area monitoring. The technology typically targets monitoring mobile patients within a hospital campus. The telemetry group feature set provides real-time data feeds, real-time arrhythmia analysis, and near real-time alarms (less than three seconds). Battery life in the patient-worn devices averages between twenty-four to forty-eight hours (using heavy and expensive nine-volt alkaline cells). The area of coverage is usually restricted to a campus, but is sometimes extended to nearby hospitals and clinics via dedicated T1 and T3 telephone lines, and occasionally line-of-sight wireless bridges.

In-home patient monitoring systems include solutions that can be categorized as home-based telemedicine programs. The telemedicine group of systems is aimed at taking a limited set of vitals and transferring these to a base station to be forwarded via wired telephony to a single repository at a doctor's office or hospital. These systems are costly, bulky, and do not follow the patient everywhere. These systems can not be considered “always ON” systems. Typically, the monitoring patient goes to the monitoring station once or twice a day and works with the equipment to take a set of vitals that are then transferred back to a database.

Improvements to these systems still require up to three wireless hops to get data back to the central station, and battery life is usually less than twenty-four hours. In addition, the sensor normally needs to be discarded upon battery exhaustion, which creates an ongoing expense over the lifetime of the system. Proposed next generation systems aim to use a single server to host connection to roaming client devices, however, these systems have not appeared on the market.

What is required is a medical monitoring system that can function both as a loop recorder and an event recorder, provide the adaptability necessary to allow one device to perform multiple functions.

Accordingly, what is required is a medical monitoring system that includes the following features:
1) A multi-function device incorporating the capabilities of several other devices including the Holter recorder, event/loop recorder, and BP monitor/recorder.
2) Multi-channel recordings (standard of 3 channels for cardiac units with extensions for up to 8 sensors).
3) 48-hour+continuous recording time in Holter mode
4) 30 day operation and recording in loop/event mode
5) 30 days+in bio statistics mode
6) Low power consumption (48 hour battery life minimum, 168 preferred)
7) Industry-standard sampling rates (120, 128, 180, 192, 240 and 256 SPS)
8) Fetal heart-rate capability on at least one channel (channel 3)
9) Pacemaker pulse detection on at least one channel (channel 3)
10) Date/Time stamped recordings
11) Patient-activated event marker with un-obscured data
12) Ability to record patient's voice (during an event) to eliminate the need for a separate patient diary
13) Built-in LCD Display for easy hookup, configuration, and patient data verification eliminating the need for a separate set of hookup channels and a separate display method
14) Upload and download capabilities (using industry standard interfaces such as USB 2.0 with a FAT file architecture and/or wireless channels)
15) Easy use for patient and clinician (self-calibrating, self-centering display/data, audio feedback, self-programming and configuring via automatic sensor detection/sensor signatures, and minimal steps to set up and use a unit)
16) High data integrity
17) Patient and data security/privacy

### SUMMARY OF THE INVENTION

Accordingly, the above requirements define the aspects of the design needed to provide the functionality for each of the listed key features.

### Multi-Function Device

The device contains the logic and software required to enable the device to provide the services of several other separate devices. The unit self-configures and adapts the user interface and behaviors based on the sensor configuration the user pairs with the unit.

For instance, pairing a 7-lead cardiac lead-set automatically places the unit into a “Holter recorder” behavior pattern. Pairing the unit with a 7-lead with communications channel lead-set causes the unit to adopt Event/loop recorder behaviors, and so on.

### Multi-channel Recordings

The multi-channel recording requirement is met by the implementation of three analog channels (extendable to eight). Each channel is independently capable of sampling and amplifying external data received through the patient lead/sensor set.

In cardiac mode, each channel accepts 60 Hz common-mode signals with common mode noise rejection to 60 db with a 1 volt peak-to-peak measurement capability.

### Long-term Continuous Recording Time (48+ hours)

The long-term recording requirement (on a single battery installation) is met by designing in a Compact Flash+ socket. The socket accommodates various sized ATA flash drives for the non-volatile storage of data. The current implementation supports up to 2 gigabyte drives.

A 256 MB compact flash provides sufficient capacity to store up to 48 hours of 3-channels un-compressed patient data including space for voice annotations. The formula used to determine capacity is: 3 channels*(256 samples/second/channel)*(60 seconds*60 minutes*24 hours). A 512M compact flash provides capacity for 168 hours (7 days). Larger flash cards can be used for longer recording periods. The industry demands 48 hours of continuous full-disclosure operation, this unit extends that requirement by providing for up to 168 hours (a planned arbitrary limit).

### 30 Day Operation in Bio Statistics Mode

Long-term operation of the device is possible because the unit can store vast amounts of data in flash. In Bio Statistics and Event/loop recorder modes, the unit uses a low-power sampling technique that allows the battery power to be conserved. When “trigger” parameters are observed during sampling, the unit then stores a sample record in permanent storage reducing the duty cycle to external storage components and thus lowering power consumption/battery drain.

To obtain periods of operation beyond the continuous life of a battery set, the unit is also designed to detect low batteries and signal a battery change to the user. Replacing the batteries is simple and straight-forward. The unit automatically resumes recording when the batteries have been replaced—no setup or data entry is required. Using internal state machines and non-volatile storage, the unit tracks what is going on even as the batteries are replaced.

An issue that falls out of this functionality is a gap in the recording. This gap is handled via time/date stamping and the 2-minute record format. See 0 Date/Time Stamped Recordings for more information on this feature.

### Low Power Consumption

The unit is designed using the latest available low-power CMOS and bi-CMOS components. The unit also has the capability of powering down various hardware sections when they are not actively in use.

This design constraint allows:
a) software to make optimum use of the available battery power,
b) enables the unit to meet the low power requirement, and
c) enables the unit to meet the long recording time requirement.

The KEY low power technique centers on a multi-stage data-pipeline data-storing algorithm. In the first stages, data is stored in on-die and on-chip ram. Subsequent stages allow the off-chip ram and compact flash to remain in powered down/hibernation states for long periods of time. The third stage storage is an ultra low-power bi-CMOS ram and is powered up occasionally to accept a data dump from on-chip ram as these buffers reach capacity. Finally, on long term intervals (once every 20 seconds) the compact flash is powered up to take ram data and commit the data to permanent storage. This multi-tier arrangement allows for the compact flash and external ram to spend more than 99% of their time in deep low-power cycles drawing mere nanoAmps of power.

Other low-power design techniques include management of the LCD display, management of the communications channels, and the use of high value pull-up and pull-down resistors in the 1M range (as opposed to the industry norm of 10K to 50K) to reduce current consumption/leakage current. The USB and real-time clock components are each driven by their own crystals. Independent crystals allow devices to be shut down when not in use to conserve power. A processor core was selected that contains a segmented architecture to allow various stages of the core to be powered down when not actively in use. Finally, a multi-stage clock is used for the main core in order to slow and sleep the main processor core between sample cycles further conserving power.

### Industry-Standard Sampling Rates

The industry standard sampling rates of 120, 128, 180, 192, 240, and 256 samples per second per channel are supported. These rates can also be stated as a sample stored every unit of time (for example: 7.8125 mSec=1.0000 second/128 samples/second).

### Fetal Heart-Rate Capability on at Least 1 Channel

Fetal heart rates are in the 180-220 beats per minute range and have wide variation. Standard filters normally reject much of this information as noise. Channel 3 in this design has increased the frequency response characteristics that allow for successful fetal heart recordings in addition to pace pulse detection/analysis (covered in the next section).

### Pacemaker Pulse Detection

Pacemaker pace pulsing can produce small variations in rhythm and the pace pulses themselves can be as short as ˜1.0 mSec in duration.

Given the lower 7.8125 mSec sampling rate described above and the obviously potentially short pace pulses of 1.0 mSec, a need to capture and report these conditions during a sample interval exists.

The way the pace pulse detection requirement is met is by using a well-known data communications and test equipment technique known as over-sampling. That is, the unit samples at a frequency much higher than the rated 128 samples per second. In this design, the sampling rate is 4096 samples/sec or a sample every 244 uSec (approximately 4 samples/1 mSec or about 30 samples per 7.8 mSec).

By taking samples of data at more frequent intervals, the unit can observe the minimum and maximum values occurring during a single storage interval of 7.8125 mSec. The storing of the values and indications of events can thus be “peak picked”. When multiple samples are combined with statistical running average and trend analysis algorithms, a more thorough picture can be ascertained as to what was actually happening to the heart rhythm as data was being obtained and stored. This signal processing is something that present-day competing recorder/monitors do not do.

Note: It should be noted that competing devices tend to reduce their sampling rates and channel counts in order to conserve power and thus reduce data quality. By sampling only once or twice per 7.8 mSec interval, some vendors have effectively conserved battery life but at the expense of the data quality and increasing missed pace pulses (except for 1 or 2 vendors, most portable device makers do not claim pace-pulse detection). This lower sample rate trade-off also causes the data collection/analysis to be statistically less accurate and currently prevents step-down cardiac patient care from using these devices to track patients and their therapy.

### Date/Time Stamped Recordings

A real-time clock is included as part of this design. The real-time clock allows for accurate time and date stamping of a recording. The clock also allows for the time stamping and correlation of any events that occurred during the recording period.

To improve data reliability, security, and design flexibility this version of the device adopts a 2-minute record size. Other record sizes could be used, but the 2-minute record has been chosen as the currently preferable record size within the industry. In turn, the 2-minute records are stamped with the date, time, recorder serial number, software version number, and unique patient mark information. This record also includes a CRC on the 2-minutes of recorded data to ensure data integrity and storage accuracy.

Breaking lengthy recordings into 2-minute segments achieves several important things. First, it matches the records up with a standard interval that doctors know and are comfortable with. Second, the segment method allows for loop or event-type recordings to be stored and properly accounts for gaps in the stored records. Lastly, the 2-minute segmentation allows for any faulty records to be detected and discarded from a Holter analysis without affecting the quality of the recording AND provides the doctor with good data even if part of the data/equipment failed eliminating the need to send the patient back out again with another recorder (which happens all too frequently with the current devices on the market—one blip and the whole recording/session is invalidated).

### Patient Activated Event Marker

A patient-activated event marker is another requirement for this unit. The way this requirement is met is by the use of a momentary contact push-button switch on the unit causing a unique hardware interrupt.

Once the unit is in cardiac data recording mode the event button can be recognized. By depressing the event button, the hardware and software handles a unique interrupt. The event interrupt causes a see-thru marker/cursor to appear in the recording. The marker is made via modulation of the sample data on all three cardiac data channels at 32 Hz.

In order to minimize the distortion of the data leading up to an event or the event itself, the recording of the event marker is delayed for 10 seconds (this means that the event itself precedes the event marker in the recording). Ten seconds (or other value selected in the setup panel) after the event button is depressed the incoming data is modulated for 2 seconds at 32 Hz. The modulation technique allows the data to remain distinguishable while making the event marker obvious.

### Ability to Record Patient's Voice

A separate and additional analog sampling channel is built into the unit. This channel is routed out to the patient lead/sensor set and provides data from a low-profile microphone. This voice channel allows for the recording of the patient voice during a cardiac event.

An omni-directional low-gain microphone is implemented as part of the patient-worn lead/sensor set. When present, the microphone makes voice data available to the analog voice channel for recording. Note that the voice data channel only opens under software control for 20 seconds after the event button has been depressed. An audible tone (piezzo device) is emitted to indicate recording is ON and another tone is emitted to indicate that recording is OFF.

Preliminary research indicates that no wire-tap or privacy laws are violated since the user knows that there is a recording going on and there is an audible note of recording. The patient lead/sensor set (and the microphone) are worn on the skin and the setup is typically hidden beneath clothing and thus could be a cause for concern. The short-duration recording of 20 seconds is long enough to obtain key diary information yet short enough to be of a lesser concern.

### LCD Display

An LCD display is provided for multiple purposes and exists for many reasons, the most important of which is user demand. Users have demanded a screen to provide an easy means of hookup, configuration, and patient data verification.

An LCD display allows the clinician to view and optionally modify (using the 5-button keypad) the patient identification data. The display further allows the clinician to verify configuration settings and patient hookup including the quality of the hookup and placement of the patient lead/sensor set.

### Upload and Download Capability

The requirement of upload and download capabilities are provided for in many ways. The first is the removable compact flash socket. By using a socketed compact flash, the unit allows the flash device to be removed and installed in another system with a compatible flash socket (such as the SanDisk Image Mate) and software driver. This technique also allows for the removable and archiving of an original record permanently.

The second upload/download mechanism built into the design is the communications channel. This design includes a multitude of communications methods including a USB 1.0/2.0 chipset, an RS-232 interface, and a wireless modem chipset (GSM or 1XRTT).

By removing the patient sensor set and replacing it with a USB transport cable, one can upload or download data to the unit. The unit connects as an endpoint only and complies with USB 1.1 and USB 2.0 Bulk Transfer specifications. The USB as implemented is capable of an average transfer rate of up to 480 mb/Sec (a 24-hour recording takes approximately 30-40 seconds to complete).

To further make the device acceptable and usable in the market, the widely accepted FAT-16 file format is used on the compact flash media. This means that a flash card can be removed and easily analyzed by any Windows-based software as long as the application has knowledge of the internal file record organization. This makes the device attractive for OEM, channel sales, and VARs.

When using a sensor set with a communications channel, the unit uses the channel to communicate (in near real-time) the data obtained from the sensors with another entity (typically a server or patient electronic record system).

### Easy Use for Patient and Clinician

By combining an LCD display, a color-coded sensor set, a 5-button keypad, and a color-coded event button the unit is fairly straightforward and can be used with minimal to no training.

The LCD display is used to guide the clinician through the step-by-step setup, patient hookup, and hookup verification procedure, By following the easy on screens prompts, the clinician is assured a smooth and accurate hookup.

The unit is self-calibrating and contains self-centering logic to center displays (such as the heart rhythm) to the center of the op-amp range.

The behavior and functionality delivered is automatically determined via another analog channel that detects and analyzes the presence of a sensor set. The sensor sets each contain a unique signature so that the behavior of the device and the human interface can be altered to match the capabilities of the sensors associated with the unit. For example: associating the USB places the unit into upload/download mode and causes the unit to appear as a data repository with a FAT file system. Associating a 7-load Holter sensor set causes the unit to start up the self-calibration and patient hookup dialogue.

All of these features together cause the device to assume behaviors based upon implied desired function. This eliminates user setup options and eliminates setup/user errors in that the unit only behaves according to the external sensors present. This means that in order to perform a successful cardiac hookup then, that a cardiac analysis sensor set must be present and associated with the device.

### High Data Integrity

Data integrity in this device is very high. A CRC per 2-minute record is generated to validate each section of the recording. Without a valid CRC, the 2-minute record is considered invalid and can be discarded. The date/time stamp present on the record also serves to identify exactly which part of the record is no good.

The validation record also contains other useful data that correlates data with device, patient, and session. The record contains a unique patient mark, device serial #, device software revision, and date/time stamp of the session.

The main log file contains a copy of the hookup information for each patient. Then, each 2-minute record in the data file contains the same marks except for the date/time stamp that moves forward in real-time time. This final step ensures that a given patient was hooked up to a known device on a given date/time and that all the records in the file match up with the unique stamp.

Any failure to erase flash records or any partial flash write/erase failures are easily detected with this scheme. Upload/download faults can also be detected and potentially recovered. The scheme provides the ability for an application to stitch together records in a seamless manner and ensure that no data loss has occurred (that can not be accounted for). It also ensures that all records being retrieved are truly part of the desired patient/session.

### Patient and Data Security/Privacy

The data contained in the device and on the flash are pure numerical data. There is no correlation between a patient's identity and the data stored on the device. The patient mark scheme used in the recorder is a free-form numbering system and is determined by the end-user. In order to be meaningful, the patient mark used in the recorder needs to be externally reconciled with a physical patient and patient name. Actual patient ID information such as SSN, name, address, phone number or other identifying marks ARE NOT contained/stored in the unit. No sort of patient identifiable information is transmitted in any form.

When used with appropriate clinical practices, this device conforms to current HIPAA privacy and security regulations.

### BRIEF DESCRIPTION OF THE SEVERAL VIEWS OF THE DRAWINGS

These and other features, aspects and advantages of the present invention will become better understood with reference to the following description, appended claims, and accompanying drawings where:

FIG. 1 is the Multi-State Data Pipelining Diagram.

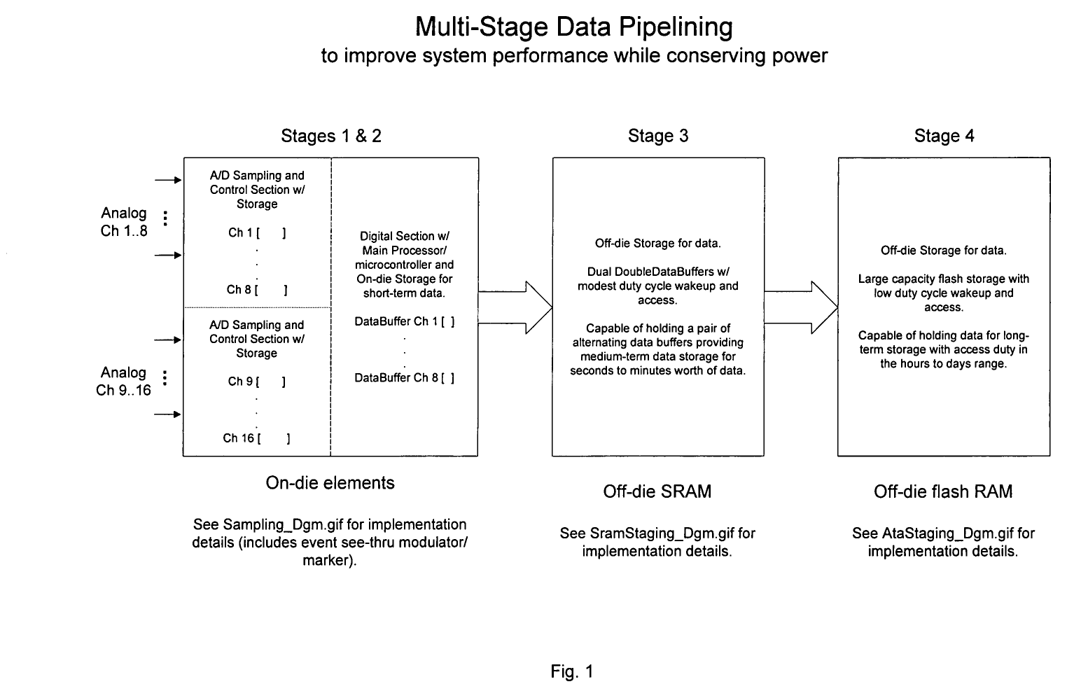

FIG. 2 is the OnChip Data Diagram.

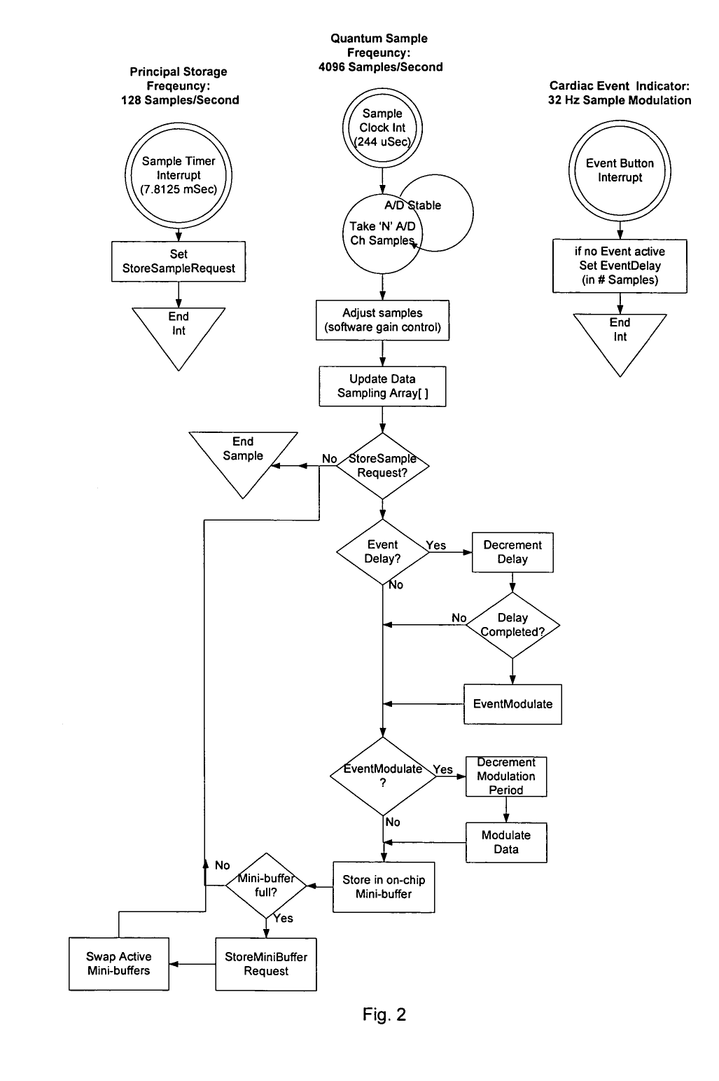

FIG. 3 is the Promotion to Off-Chip RAM Diagram.

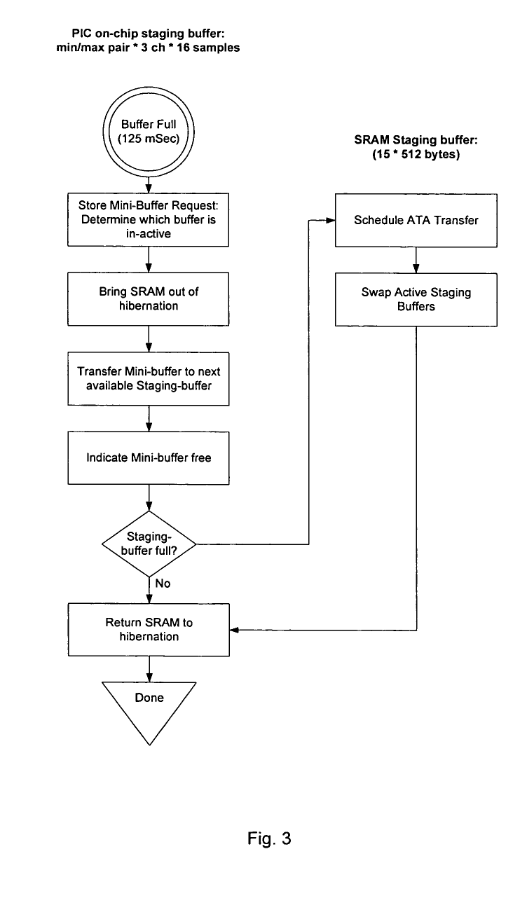

FIG. 4 is the Promotion to Permanent Storage Diagram.

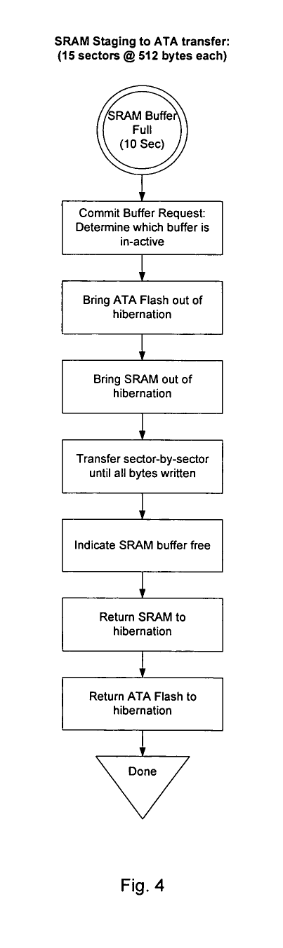

FIG. 5 is an example of a Record including an Event Marker Overlay.

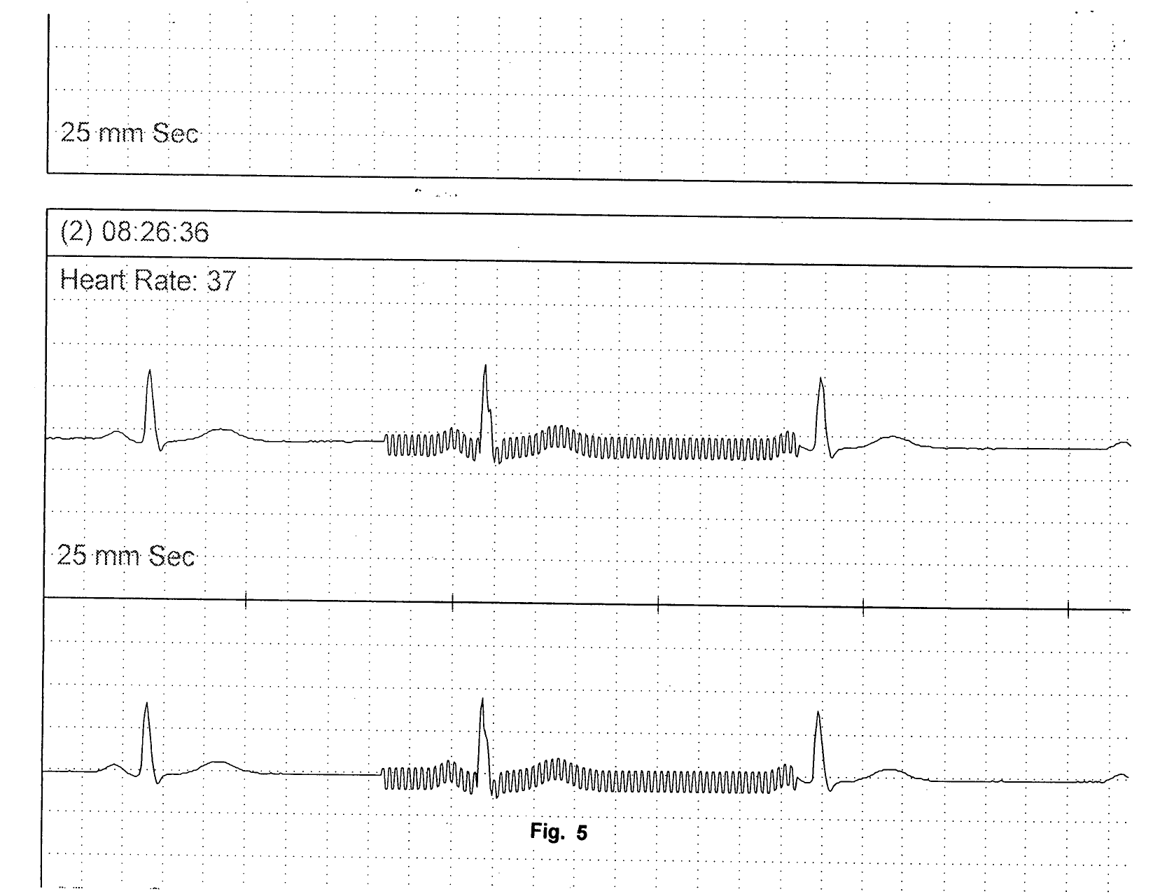

FIG. 6 is the System Level Block Diagram.

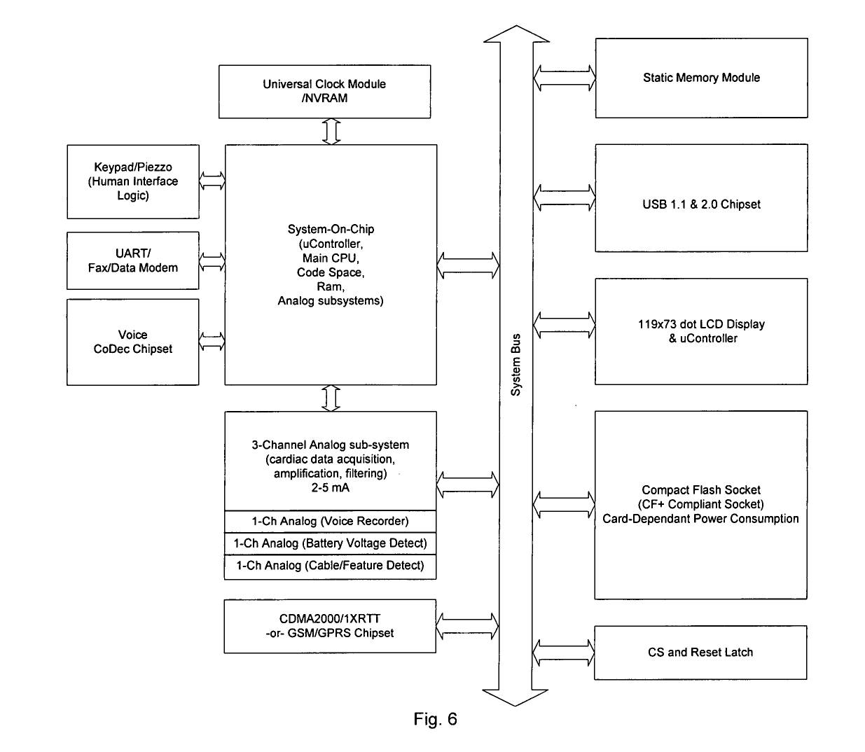

### DETAILED DESCRIPTION OF LOGICAL SYSTEM FLOW

This section outlines the general flow of data recording through the system from the bottom up.

### Analog Inputs

The lowest portion of the system design is the analog data interface. The system is designed to use up to sixteen analog channels. The channels are broken into two groups of eight channels each.

Group 0—Bio Sensor Channels

Data acquisition is very flexible. The system presently reports on 8-bit quality although it collects 10-bit resolution samples. The system is also capable of 12-bit resolution. Currently no resolution higher than 8 bits is reported since all current software and patient data models are centered on 8-bit data repositories. The group is capable of high-rate sampling.

The first eight analog channels are grouped together on one A/D controller interface and are dedicated to real-time collection of data from the biometric sensors. These bio sensor channels are designed to amplify and filter the incoming signals to an appropriate quality for recording. Note that filtering does not mean to alter, distort, or change the general shape of the waveform. Filtering in this sense means rejecting outside noise and impulses.

Group 1—System-Level Channels

The second group of channels has a lower acquisition resolution of a fixed 8-bit quality. The resolution here is dedicated to serving low duty cycle system services. This group is restricted to low rate sampling of around 100 kHz.

One analog channel in this group is dedicated as part of an amplifier/acquisition circuit designed to be compatible with a low-gain, omni-directional microphone that is used to record the patient voice as part of the diary feature.

The second channel in the group is used to track battery power level and consumption.

The last channel is implemented to track and identify the associated sensor set and control the device behaviors. Various unique signatures are assigned to each sensor set. This signature in turn correlates the expected functions and behaviors with the selected sensor set.

### MicroController (uC) and On-Chip Storage

To obtain the best analysis of cardiac data and improve noise immunity, the uC continuously samples the analog channels (about once every 150 uSecs). During this acquisition cycle the interrupt thread keeps a statistical running average per channel.

Periodically, (once every 7.8125 mSec) a data storage thread wakes up via a timer service to store the data from the real-time collection bin. The thread takes the sampled analog data (12/10-bits) and normalizes it to eight bits (1× gain drops the least 2 or 4 significant bits).

A gain adjustment is built in to allow a greater swing in the A/D conversions to provide a proper 1 V peak-to-peak measurement on a differential analog input pair. The gain feature allows for compensation due to poor skin conduction, low-quality sensor sets, and other environmental factors that can affect the quality of the recording.

As the uC samples the analog data, it is stored in the on-chip buffer until it is nearly full. By storing data in the on-chip buffer the speed of data collection is maximized while power drain on the system is minimized since all support parts are kept in low-power stand-by mode. Once the on-chip buffer fills up (about once every half-second), the buffer is quickly transferred to the next stage.

### System SRAM Buffering

When the uC on-chip memory data buffer is full (256 bytes available) the SRAM is brought out of hibernation. The uC on-chip buffer is quickly transferred to the SRAM staging buffer and if the buffer has more space, the SRAM returns to hibernation. This buffer staging and transfer process repeats itself until there is sufficient data to fill an ATA flash buffer (30*512 bytes).

When the SRAM buffer contains enough sectors of data for the flash, an ATA transfer event is scheduled (this is once every 20 seconds). The ATA event moves the SRAM buffer to the internal staging area of the flash and commits that data to semi-permanent storage. The ATA event must complete prior to the filling up of the next uC on-chip buffer (256 bytes of data). This gives the uC and ATA components around 600 mSec ((256/3)*7.8125 mSec) to complete this job. Ideally, writing 30 sectors to the flash takes between 30 and 50 mSec leaving plenty of time to spare for other chores and poses no risk of data loss.

### ATA Flash Writing

When the SRAM buffer is filled to capacity (a 30 sector transfer is ready) the data buffer is moved to semi-permanent storage on the ATA flash part. This is the highest power-consuming period of the recording process as the uC, system SRAM, and ATA flash are all active simultaneously. The flash-write event is scheduled and takes many steps so code is hand-optimized to minimize path length (current consumption can be as high as 70 mA during this time).

The uC, SRAM, and flash interact approximately once every 20 seconds (30 buffers×0.5 KB/buffer) for between 30 and 50 mSec. During ATA recording the data is committed to the flash memory and the ATA card is placed back into hibernation to conserve battery power. When the ATA recording event has completed, the SRAM is no longer needed either and it too is placed back into hibernation.

This store-wake-store-hibernate process loops until the pre-determined recording period has been met.

### Other

LCD Display

The LCD display is active when needed. When active, the nominal current consumption is about 5-7 mA. Battery life is extended by shutting down the display when not in use. Depressing any interface keys or the event button causes the display to be re-enabled. The display returns to hibernation after 2 minutes of inactivity.

Communications Channels

The data modem and RS-232 channels remain in hibernation most of the time. The channels are only activated when a record needs to be transferred to another entity. Record mode scheduling and transfer is used to minimize power consumption and retain system performance.

USB Microcontroller and SIE

The USB components are in hibernation during normal cardiac data recording activity.

The USB is used only during configuration, setup, download, and upload activities. This further conserves operational period battery power.

1. A medical monitoring system comprising:
an operating mode selector for selecting a current mode;
a user interface, wherein said user interface is adaptable to said current mode;
a means for receiving a set of physiological data, wherein said means for receiving said set of physiological data comprises at least one sensor, wherein said sensor communicates with said operating mode selector;
a means for storing said set of physiological data;
a means for transmitting said set of physiological data;
a data record, wherein said data record comprises a representation of said set of physiological data;
a means for attaching a record information mark to said data record; and
a means for ensuring data integrity of said data record.

2. The medical monitoring system of claim 1, wherein said means for receiving a set of physiological data includes at least one high frequency response channel.

3. The medical monitoring system of claim 2, wherein said means for ensuring data integrity comprises a validation record.

4. The medical monitoring system of claim 3, wherein said user interface includes a graphic display.

5. The medical monitoring system of claim 4, further comprising a low power consumption system.

6. The medical monitoring system of claim 5, wherein said means for transmitting said set of physiological data includes a communications channel.

7. The medical monitoring system of claim 6, wherein said data record further comprises a record period.

8. The medical monitoring system of claim 7, wherein said means for determining an operating mode includes a plurality of sensors.

9. The medical monitoring system of claim 8, wherein said means for determining an operating mode includes a sensor detector.

10. The medical monitoring system of claim 9, wherein said record period is substantially two minutes in duration.

11. The medical monitoring system of claim 10, wherein said means for stamping a record information mark on said data record includes a real-time clock.

12. The medical monitoring system of claim 11, wherein said record information mark comprises: a date, a time, a recorder serial number, a software version number, and a patient identification mark.

13. The medical monitoring system of claim 12, wherein said user interface further comprises at least one push button.

14. The medical monitoring system of claim 13, wherein said user interface further comprises an event button.

15. The medical monitoring system of claim 14, further comprising an event condition, wherein said event condition is evidenced by an activation of said event button.

16. The medical monitoring system of claim 15, wherein said activation of said event button creates an event marker.

17. The medical monitoring system of claim 16, wherein said event marker comprises a substantially transparent overlay, wherein said substantially transparent overlay is located on said data record.

18. The medical monitoring system of claim 17, wherein said event marker is created after an event condition delay.

19. The medical monitoring system of claim 18, wherein said event condition delay is approximately ten seconds in duration.

20. The medical monitoring system of claim 19, further comprising a plurality of voice data channels.

21. The medical monitoring system of claim 20, wherein said plurality of voice data channels includes a first voice data channel, and wherein said plurality of voice data channels includes a second voice data channel.

22. The medical monitoring system of claim 21, wherein said first voice data channel is capable of allowing a first communication, wherein said first communication includes a health service provider and wherein said first communication includes a patient.

23. The medical monitoring system of claim 22, wherein said second voice data channel comprises a patient voice recorder.

24. A medical monitoring system comprising:
an operating mode selector for selecting a current mode, wherein said operating mode selector includes a plurality of sensors, and wherein said operating mode selector includes a sensor detector;
a user interface, wherein said user interface comprises at least one push button, and wherein said user interface further comprises an event button, and wherein said user interface includes a graphic display;
a means for receiving a set of physiological data, wherein said means for receiving said set of physiological data includes at least one high frequency response channel;
a means for storing said set of physiological data;
a means for transmitting said set of physiological data, wherein said means for transmitting said set of physiological data includes at least one communications channel;
a data record, wherein said data record comprises a representation of said set of physiological data;
a means for ensuring data integrity of said data record, wherein said means for ensuring data integrity comprises a validation record.
